(19) United States
(12) Patent Application Publication
(10) Pub. No.: US 2026/0360558 A1
(43) Pub. Date: Nov. 12, 2026

(54) RHEUMATOID ARTHRITIS-ASSOCIATED INTERSTITIAL LUNG DISEASE PROGRESSION RISK PREDICTION SYSTEM

(71) Applicant: Soonchunhyang University Industry Academy Cooperation Foundation, Asan-si (KR)

(72) Inventors: Sung Hae Chang, Seoul (KR); EUNYOUNG LEE, Seoul (KR); JUNG YOON CHOE, Daegu (KR); YONGBEOM PARK, Seoul (KR); YEON-AH LEE, Seoul (KR); EUN HA KANG, Seongnam-si (KR)

(73) Assignee: Soonchunhyang University Industry Academy Cooperation Foundation, Asan-si (KR)

(21) Appl. No.: 19/674,001

(22) Filed: May 11, 2026

(30) Foreign Application Priority Data
May 9, 2025 (KR) 10-2025-0060604

(51) Int. Cl. G16H 50/30; G16H 10/20

(52) U.S. Cl. CPC G16H 50/30; G16H 10/20

(57) ABSTRACT

An RA-ILD progression risk prediction system according to the present disclosure includes a data input unit configured to receive serological test data and clinical test data of a target person group belonging to a predefined cohort for constructing a prediction model, and receive serological test data and clinical test data of a predicted target person, a preprocessing unit configured to perform preprocessing of the serological test data and the clinical test data, a risk score calculation unit configured to apply preprocessed serological test data and preprocessed clinical test data of the predicted target person to a pre-constructed prediction model to calculate an RA-ILD progression risk score of the predicted target person, and a classification unit configured to classify the predicted target person into one of a preset risk section based on the RA-ILD progression risk score.

### CROSS-REFERENCE TO RELATED APPLICATION

This application claims priority under 35 U.S.C. § 119 (a) to Korean Patent Application No. 10-2025-0060604, filed on May 9, 2025, with the Ministry of Intellectual Property of the Republic of Korea, the disclosure of which is incorporated herein by reference in its entirety.

### BACKGROUND

The present disclosure relates to a rheumatoid arthritis-associated interstitial lung disease (RA-ILD) progression risk prediction system, and more particularly, to an RA-ILD progression risk prediction system which quantitatively calculates disease progression possibility based on a patient's anti-cyclic citrullinated peptide (anti-CCP) antibody titers, lung function indicators, image-based fibrosis patterns, and lung damage biomarkers, and classifies the patient into one of a low-risk group, a moderate-risk group, and a high-risk group based on calculated prediction scores.

Interstitial lung disease (ILD) is a chronic disease in which fibrosis occurs in lung tissue due to various causes, and in some patients, a pattern of progressive pulmonary fibrosis (PPF) appears, in which fibrosis worsens over time.

PPF is presented as a broader concept of progressive fibrosis that may be observed in various forms of ILD without being limited to IPF, and clinical importance thereof has rapidly increased, particularly following the introduction of antifibrotic therapies.

In fact, PPF is used as a patient inclusion criterion in numerous clinical trials for therapeutic agents, and accordingly, whether there is PPF serves as a threshold for initiating treatment and a criterion for clinical decision.

However, despite the widespread clinical application of the concept of PPF, there is still a lack of unified definitions or evaluation criteria for diagnosing or predicting the PPF.

Such ambiguity causes confusion in decision-making during actual clinical application and hinders the assurance of consistency in study design.

In this regard, the American Thoracic Society (ATS), the European Respiratory Society (ERS), the Japanese Respiratory Society (JRS), and the Latin American Thoracic Society (ALAT) presented a standardized definition of PPF through a joint guideline in 2022, and the standardized definition is based on three core areas: worsening of respiratory symptoms, deterioration of pulmonary physiological indicators, and progression of imaging findings.

Nevertheless, the study to apply the standardized definition of PPF to actual clinical practice are rare, and in particular, the study for evaluating or predicting disease progression based on PPF criteria for RA-ILD are extremely limited.

RA-ILD is commonly classified as a subtype of autoimmune rheumatic disease-associated ILD (SARD-ILD), but in reality, RA-ILD exhibits pathophysiological characteristics and clinical courses that are clearly distinct from other forms of SARD-ILD.

In other words, compared to other SARDs, RA-ILD is predominantly characterized by imaging findings of a usual Interstitial pneumonia (UIP) pattern, and is known to resemble IPF and associated with a severe prognosis and high mortality. Also, various studies have suggested that RA-specific arthritis activity, systemic inflammation, and autoantibody production may have a complex impact not only on the onset of ILD but also on potential progression.

Nevertheless, a clear analysis of causal relationship between RA disease activity and ILD progression is still lacking, which acts as a limitation in risk prediction or establishment of early intervention strategies specific to RA-ILD patients.

In particular, most known studies have underestimated the contribution of rheumatoid arthritis (RA)-specific factors by integrating and analyzing ILD as a whole, and accordingly, there is a lack of quantitative prediction means or objective risk classification systems applicable to the RA-ILD patient group.

The related art includes Korea Patent No. 10-2710842 (published on Sep. 27, 2024).

### SUMMARY

Provided is a rheumatoid arthritis-associated interstitial lung disease (RA-ILD) progression risk prediction system which quantitatively calculates disease progression possibility based on a patient's anti-cyclic citrullinated peptide (anti-CCP) antibody titers, lung function indicators, image-based fibrosis patterns, and lung damage biomarkers, and classifies the patient into one of a low-risk group, a moderate-risk group, and a high-risk group based on calculated prediction scores.

Additional aspects will be set forth in part in the description which follows and, in part, will be apparent from the description, or may be learned by practice of the presented embodiments of the disclosure.

According to the present disclosure, a rheumatoid arthritis-associated interstitial lung disease (RA-ILD) progression risk prediction system includes a data input unit configured to receive serological test data and clinical test data of a target person group belonging to a predefined cohort for constructing a prediction model, and receive serological test data and clinical test data of a predicted target person, a preprocessing unit configured to perform preprocessing of the serological test data and the clinical test data, a risk score calculation unit configured to apply preprocessed serological test data and preprocessed clinical test data of the predicted target person to a pre-constructed prediction model to calculate an RA-ILD progression risk score of the predicted target person, and a classification unit configured to classify the predicted target person into one of a preset risk section based on the RA-ILD progression risk score.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a diagram illustrating a rheumatoid arthritis-associated interstitial lung disease (RA-ILD) progression risk prediction system according to an embodiment of the present disclosure;

FIG. 2 is a graph illustrating a cumulative incidence rate if an RA-ILD progression of a first prediction model according to an embodiment of the present disclosure; and

FIG. 3 is graph illustrating a cumulative incidence rate of an RA-ILD progression of a second prediction model according to an embodiment of the present disclosure.

### DETAILED DESCRIPTION

Hereinafter, preferred embodiments according to the present disclosure will be described in detail below with reference to the attached drawings. In this process, thicknesses of lines, sizes of components, and so on illustrated in the drawings may be exaggerated for the sake of clarity and convenience of description.

In addition, the terms described below are defined considering their functions in the present disclosure and may change depending on intention or convention of a user or operator. Therefore, definitions of the terms should be based on the content throughout the present disclosure.

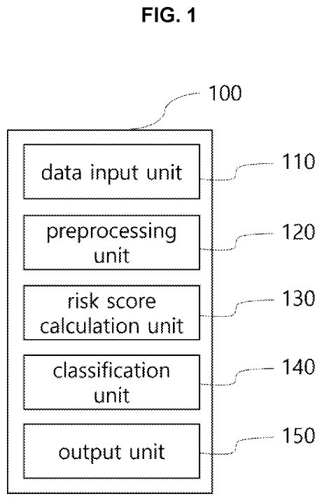

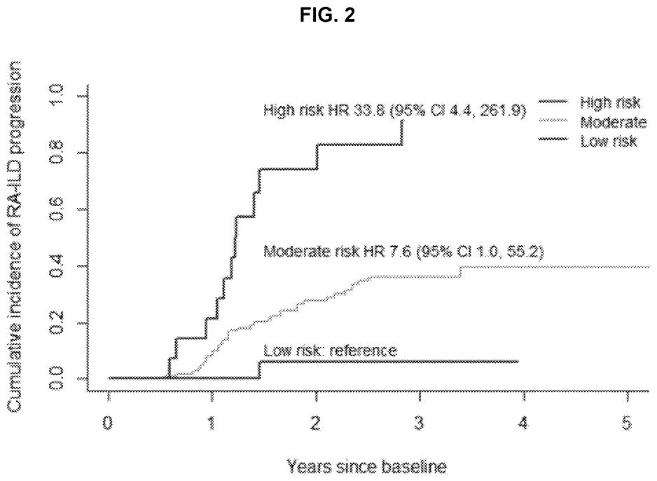

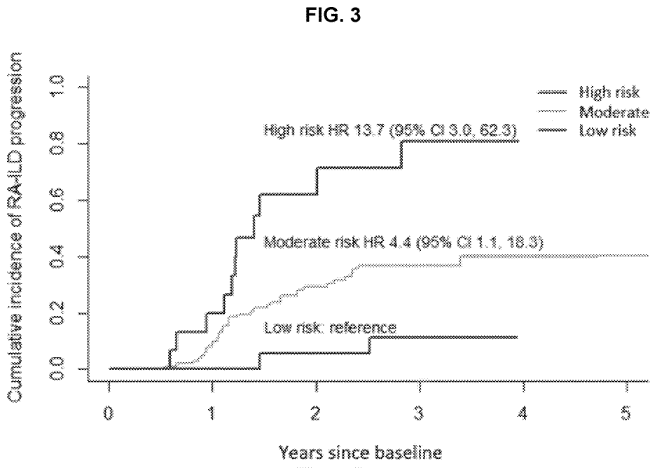

FIG. 1 is a diagram illustrating a rheumatoid arthritis-associated interstitial lung disease (RA-ILD) progression risk prediction system according to an embodiment of the present disclosure, FIG. 2 is a graph illustrating a cumulative incidence rate of an RA-ILD progression of a first prediction model according to an embodiment of the present disclosure, and FIG. 3 is graph illustrating a cumulative incidence rate of an RA-ILD progression of a second prediction model according to an embodiment of the present disclosure.

As illustrated in FIG. 1, an RA-ILD progression risk prediction system 100 according to an embodiment of the present disclosure includes a data input unit 110, a preprocessing unit 120, a risk score calculation unit 130, a classification unit 140, and an output unit 150.

The data input unit 110 receives serological test data and clinical test data of a target person group belonging to a predefined cohort for constructing a prediction model, and receives serological test data and clinical test data of a predicted target person.

In this case, the cohort consists of a group of patients registered in Korean RA-ILD cohort (KORAIL), a domestic multi-center RA-ILD cohort, and more specifically, includes adult patients aged 18 years or older who are diagnosed with rheumatoid arthritis (RA) and diagnosed with interstitial lung disease (ILD) based on chest computerized tomography (CT) images.

In addition, the serological test data may include an anti-cyclic citrullinated peptide (anti-CCP) value and at least one pulmonary damage biomarker selected from among Krebs von den Lungen-6 (KL-6), human surfactant protein D (hSP-D), and matrix metalloproteinase-7 (MMP-7).

In addition, the clinical test data includes pulmonary function test (PFT) data and chest imaging test data.

In this case, the PFT data includes diffusing capacity of the lung for carbon monoxide (DLCO) data and forced vital capacity (FVC) data.

In addition, usual interstitial pneumonia (UIP) pattern is present, an ILD extent, and involvement or severity scores of reticular opacity (RO), honeycombing (HC), ground-glass opacity (GGO), and traction bronchiectasis/bronchiolectasis (TBE).

The preprocessing unit 120 performs preprocessing of serological test data and clinical examination data.

More specifically, the preprocessing unit 120 may perform a log transformation on the serological test data to normalize a data distribution, and then remove a unit difference between input variables, and ensure that each characteristic is evenly reflected in a prediction model.

In addition, the preprocessing unit 120 may calculate a score value obtained by quantifying the degree of lesion involvement based on chest CT images for the clinical test data.

More specifically, the preprocessing unit 120 may divide the lungs into a total of six portions including a left upper portion, a left medium portion, a left lower portion, a right upper portion, a right middle portion, and a right lower portion, based on the clinical test data, and semi-quantitatively score RO, HC, GGO, and TBE lesions for each portion according to the degree of involvement based on a preset criterion from 0 to 4.

Also, the preprocessing unit 120 may classify the ILD involvement range into sectionalized categorical values depending on predefined threshold values based on the entire lung area.

For example, the ILD involvement range may be divided into a section of 10% or less, a section greater than 10% and less than 30%, and a section of 30% or more.

The risk score calculation unit 130 may apply the preprocessed serological test data and clinical test data to a pre-constructed prediction model to calculate an RA-ILD progression risk score of a predicted target person.

In this case, the prediction model may predict a likelihood of RA-ILD progression in the subject within a preset period based on Cox proportional hazards regression analysis by using an anti-CCP value, a DLCO predicted value, an ILD extent, and whether a definite/probable UIP pattern is present as input variables, and may further use a KL-6 value when the KL-6 value is provided. The prediction model may calculate the predicted likelihood in the form of a quantified risk score.

In this case, the RA-ILD progression is defined as observation of both deterioration in lung function and deterioration in imaging finding.

The deterioration in lung function is defined as a decrease in FVC by 5% or more, or a decrease in DLCO by 10% or more, relative to a reference value within one year. The present disclosure may satisfy one or more of a case where the extent or severity of lung lesions increases on follow-up chest CT and a case where RO, HC or GGO findings that are previously unobserved newly occur with concurrent traction bronchiectasis (TBE) or bronchiolectasis.

Also, the prediction model may be constructed in two forms of a first prediction model and a second prediction model depending on whether KL-6 values are provided.

The first prediction model is a prediction model in which a KL-6 value is provided, and may be implemented by Equation 1 below.

$$Total Score = 4 ( AntiCCP \geq \times 40 ULN ) + 6 ( Serum KL 6 \geq 730 U / ml ) + 7 ( ILD extent > 10 \% ) + 7.5 ( DLCO \% pred . < 55 \% ) + 8 ( \frac{Definite}{probable} UIP ) \qquad Equation 1$$

In this case, ULN is a normal upper limit, AntiCCP is an anti-CCP value, Serum KL6 is a KL-6 value, ILD extent is an ILD range based on a CT image, DLCO % pred is a predicted value of DLCO, and Definite/probable UIP is a diagnostic result of a UIP pattern.

The second prediction model is a prediction model for being replaced with the first prediction model when the KL-6 value is not provided, and may be implemented by Equation 2 below.

$$Total Score = 3 ( AntiCCP \geq \times 40 ULN ) + 4 ( \frac{Definite}{probable} UIP ) + 4.5 ( DLCO \% pred . < 55 \% ) + 5 ( ILD extent > 10 \% ) \qquad Equation 2$$

The classification unit 140 may classify a predicted target person into one of preset risk sections based on the RA-ILD progression risk score.

In this case, the risk sections may be set differently depending on the type of prediction model.

For example, when the RA-ILD progression risk score is less than 4, the first prediction model may classify the predicted target person into a low-risk group; and when the RA-ILD progression risk score is 4 or more and less than 25.5, the first prediction model may classify the predicted target person into a moderate-risk group, and when the RA-ILD progression risk score is 25.5 or more, the first prediction model may classify the predicted target person into a high-risk group.

In this case, a hazard ratio (HR) according to the risk section classified through the first prediction model is illustrated in FIG. 2. The low-risk group is set as a reference group and a relative degree of risk is considered to be 1. The moderate risk group is checked to have an HR of 7.6 (95% CI: 1.0 to 55.2), showing an RA-ILD progression risk that is about 7.6 times higher than the low-risk group. The high-risk group has the highest probability of RA-ILD progression within 5 years, with an HR of 33.8 (95% CI: 4.4 to 261.9), showing an RA-ILD progression risk that is about 33.8 times higher than the low-risk group.

In addition, the second prediction model may classify a target person into the low-risk group when the RA-ILD progression risk score is less than 3, the second prediction model may classify the target person into a moderate-risk group when the RA-ILD progression risk score is 3 or more and less than 13.5, and the second prediction model may classify the target person into a high-risk group when the RA-ILD progression risk score is 13.5 or more.

In this case, an HR according to the risk section classified through the second prediction model is illustrated in FIG. 3. The low-risk group is set as a reference group and a relative degree of risk is considered to be 1. The moderate risk group is checked to have an HR of 4.4 (95% CI: 1.1 to 18.3), showing an RA-ILD progression risk that is about 4.4 times higher than the low-risk group. The high-risk group has the highest probability of RA-ILD progression within 5 years, with an HR of 13.7 (95% CI: 3.0 to 62.3), showing an RA-ILD progression risk that is about 13.7 times higher than the low-risk group.

The output unit 150 may provide a user with the calculated RA-ILD progression risk score of a predicted target person and the classified risk section.

Also, the output unit 150 may provide a user with a clinical warning or a recommendation for treatment intervention based on the classified risk section.

As described above, according to an embodiment of the present disclosure, the possibility of disease progression may be quantitatively predicted by reflecting the pathophysiology and clinical characteristics of patients with RA-ILD. Accordingly, the present disclosure may support risk stratification of patients with RA-ILD into low-risk, moderate-risk, and high-risk groups, thereby facilitating determination of appropriate monitoring intensity and timing of treatment intervention.

Although the present disclosure is described with reference to embodiments illustrated in the drawings, the embodiments are only examples, and those skilled in the art will understand that multiple modifications and equivalent alternative embodiments may be derived therefrom. Therefore, the true technical protection scope of the present disclosure should be determined by the technical concept of the following claims.

What is claimed is:

1. A rheumatoid arthritis-associated interstitial lung disease (RA-ILD) progression risk prediction system comprising:
a data input unit configured to receive serological test data and clinical test data of a target person group belonging to a predefined cohort for constructing a prediction model, and receive serological test data and clinical test data of a predicted target person;
a preprocessing unit configured to perform preprocessing of the serological test data and the clinical test data;
a risk score calculation unit configured to apply preprocessed serological test data and preprocessed clinical test data of the predicted target person to a pre-constructed prediction model to calculate an RA-ILD progression risk score of the predicted target person; and
a classification unit configured to classify the predicted target person into one of a preset risk section based on the RA-ILD progression risk score.

2. The rheumatoid arthritis-associated interstitial lung disease progression risk prediction system of claim 1, wherein
the serological test data includes an anti-cyclic citrullinated peptide (anti-CCP) value and at least one pulmonary damage biomarker selected from among Krebs von den Lungen-6 (KL-6), human surfactant protein D (hSP-D), and matrix metalloproteinase-7 (MMP-7).

3. The rheumatoid arthritis-associated interstitial lung disease progression risk prediction system of claim 1, wherein
the clinical test data includes pulmonary function test (PFT) data and chest imaging test data,
the PFT data includes diffusing capacity of the lung for carbon monoxide (DLCO) data and forced vital capacity (FVC) data, and
the chest imaging test data includes whether there is a usual interstitial pneumonia (UIP) pattern, an interstitial lung disease (ILD) involvement range, reticular opacity (RO), honeycombing (HC), ground-glass opacity (GGO), and traction bronchiectasis (TBE) and bronchiolectasis.

4. The rheumatoid arthritis-associated interstitial lung disease progression risk prediction system of claim 1, wherein
the preprocessing unit is further configured to perform a log transformation on the serological test data to normalize a distribution of the serological test data.

5. The rheumatoid arthritis-associated interstitial lung disease progression risk prediction system of claim 1, wherein
the preprocessing unit is further configured to calculate a score value obtained by quantifying lesion involvement range based on a chest computerized tomography (CT) image on the clinical test data.

6. The rheumatoid arthritis-associated interstitial lung disease progression risk prediction system of claim 1, wherein
the preprocessing unit is further configured to classify an ILD involvement range into sectionalized categorical values depending on predefined threshold values based on the entire lung area.

7. The rheumatoid arthritis-associated interstitial lung disease progression risk prediction system of claim 1, wherein
the prediction model is constructed to predict a likelihood of RA-ILD progression in the subject within a preset period based on Cox proportional hazards regression analysis by using an anti-CCP value, a DLCO predicted value, an ILD extent, and whether a definite/probable UIP pattern is present as input variables, and further using a KL-6 value when the KL-6 value is provided, and to calculate the predicted likelihood as a quantified risk score.

8. The rheumatoid arthritis-associated interstitial lung disease progression risk prediction system of claim 7, wherein
the prediction model includes two forms including a first prediction model and a second prediction model depending on whether the KL-6 value is provided.

9. The rheumatoid arthritis-associated interstitial lung disease progression risk prediction system of claim 8, wherein
the first prediction model is a prediction model when the KL-6 value is provided, and is implemented by an equation below,
$$Total Score = 4 ( AntiCCP \geq \times 40 ULN ) + 6 ( Serum KL 6 \geq 730 U / ml ) + 7 ( ILD extent > 10 \% ) + 7.5 ( DLCO \% pred . < 55 \% ) + 8 ( \frac{Definite}{probable} UIP )$$
where ULN is an upper limit of normal, AntiCCP is an anti-CCP value, Serum KL6 is the KL-6 value, ILD extent is an ILD range based on a CT image, DLCO % pred is the DLCO prediction value, and definite/probable UIP is a diagnostic result of the UIP pattern.

10. The rheumatoid arthritis-associated interstitial lung disease progression risk prediction system of claim 8, wherein
the second prediction model is a prediction model when the KL-6 value is not provided, and is implemented be an equation below,
$$Total Score \geq 3 ( AntiCCP \geq \times 40 ULN ) + 4 ( \frac{Definite}{probable} UIP ) + 4.5 ( DLCO \% pred . < 55 \% ) + 5 ( ILD extent > 10 \% )$$
where ULN is an upper limit of normal, AntiCCP is an anti-CCP value, ILD extent is an ILD range based on a CT image, DLCO % pred is the DLCO prediction value, and definite/probable UIP is a diagnostic result of the UIP pattern.

11. The rheumatoid arthritis-associated interstitial lung disease progression risk prediction system of claim 1, further comprising:
an output unit configured to provide a user with a calculated RA-ILD progression risk score of the predicted target person and a classified risk section.

12. The rheumatoid arthritis-associated interstitial lung disease progression risk prediction system of claim 1, wherein
the output unit is further configured to provide the user with a clinical warning or a treatment involvement recommendation depending on the classified risk section.
